(19) United States
(12) Patent Application Publication
(10) Pub. No.: US 2025/0325594 A1
(43) Pub. Date: Oct. 23, 2025

(54) METHOD FOR INDUCING TISSUE REGENERATION AND TREATING AUTOIMMUNE DISEASES USING MESENCHYMAL STEM CELLS

(71) Applicant: REGROW BIOSCIENCES PRIVATE LIMITED, Pune (IN)

(72) Inventors: Satyen SANGHAVI, Pune (IN); Vinayak KEDAGE, Pune (IN)

(21) Appl. No.: 19/184,352

(22) Filed: Apr. 21, 2025

(30) Foreign Application Priority Data
Apr. 22, 2024 (IN) 202421031908

(51) Int. Cl. A61K 35/28; A61K 9/00; A61K 38/18; A61K 38/20; A61P 19/02

(52) U.S. Cl. CPC A61K 35/28; A61K 9/0019; A61K 38/1866; A61K 38/2066; A61P 19/02

(57) ABSTRACT

The present disclosure relates to a method of inducing tissue regeneration, said method comprising administering to said tissue a composition comprising a population of mesenchymal stem cells (MSCs) and a carrier, wherein said composition possess an inherent ability to reduce inflammation by increasing levels of IL-10 and decreasing levels of TNF-α and IL-6. The present disclosure also relates to a method of treating an autoimmune disease through regeneration of cartilage and bone tissues in a subject, the method comprising: administering therapeutic amount of a composition comprising a population of mesenchymal stem cells (MSCs) and a carrier to the subject by intra-plantar route, intravenous route, intramuscular route, intraarticular route, intraosseous route, or subcutaneous route; wherein said population of MSCs is a homogeneous population having a size in the range of 15-30 μm; and wherein said composition comprises secretome of said MSCs having VEGF and IL-10.

### RELATED APPLICATION

The present application claims priority to Indian Patent Application number 202421031908, filed on Apr. 22, 2024, which is incorporated herein by reference in its entirety.

### FIELD OF INVENTION

The present disclosure relates to the field of mesenchymal stem cells (MSCs) and, particularly to, a method of inducing tissue regeneration using a composition comprising MSCs. The present disclosure further relates to a method of treating autoimmune disease using the composition.

### BACKGROUND OF THE INVENTION

Rheumatoid arthritis (RA), an autoimmune disease, is the most common inflammatory joint disorder found in most of the countries in world where every year many more are diagnosed for this disease. Worldwide, the annual incidence of RA is approximately 3 cases per 10,000 population, and the prevalence rate is approximately 1%, increasing with age and peaking between the ages of 35 and 50 years (Howard R Smith and Adam Brown. What is the global prevalence of rheumatoid arthritis (RA) among different age groups and ethnicities? Rheumatoid Arthritis (RA) Questions & Answers February 2020: URL: https://www.medscape.com/answers/331715-5335/what-is-the-global-prevalence-of-rheumatoid-arthritis-ra-among-different-age-groups-and-ethnicities #:˜:text-Worldwide % 2C %20the %20annual %20incidence %20of,of %20 35% 20and %2050%20 years.) RA is a chronic systemic inflammatory disorder usually affecting the symmetrical bilateral joints. Uncontrolled RA characterized by progressive damages to synovial, cartilage, and bone is associated, probably, with extra articular signs. Due to chronic action of RA throughout the entire body which may lead to joint destruction and eventual deformity (Scott D L, Wolfe F, Huizinga T W. Rheumatoid arthritis. Lancet 2010; 376 (9746): 1094-1108 DOI 10.1016/S0140-6736 (10) 60826-4). The potential pathogenic mechanism of RA initiation and its development include genetic-environmental interactions, synovial immunologic processes and inflammation resulting in loss of immunological self-tolerance (Wang L, Wang L, Cong X, Liu G, Zhou J, Bai B, Li Y, Bai W, Li M, Ji H, Zhu D, Wu M, Liu Y. Human umbilical cord mesenchymal stem cell therapy for patients with active rheumatoid arthritis: safety and efficacy. Stem Cells and Development 2013; 22 (24): 3192-3202 DOI 10.1089/scd.2013.0023). RA is mostly affecting multiple joints very commonly with smaller peripheral joints (e. g. fingers, toes, thumbs, wrists). Further, this disease tends to be symmetrical in nature such that those who suffer from the disease experience symptoms in both hands and both knees, however there was some known exceptions (Alasdair G. Kay, Jim Middleton, Oksana Kehoe. Mesenchymal stem cell therapy in Rheumatoid Arthritis 2016; DOI: 10.1007/978-3-319-40144-7_8). Treatment options are limited and mainly focus on symptomatic relief rather than developmental delay or halting the progression of disease. Inflammation caused by RA are managed using nonsteroidal anti-inflammatory drugs (NSAIDs), standard pain killers, analgesics, disease modified antirheumatic drugs (DMAIDS). Recent research for novel treatment has led to the introduction of new biological agents in RA management such as anti-tumor necrosis factor alpha (anti-TNF-α), anti-interleukin 1 (IL-1) and anti-interleukin 6 (IL-6). However, these are also found to be of limited significance as RA development and progression has multifaceted etiology with the interaction of a complex combination of antigens and biological signaling molecules leading to the various symptoms of the disease (Alasdair G. Kay, Jim Middleton, Oksana Kehoe. Mesenchymal stem cell therapy in Rheumatoid Arthritis 2016; DOI: 10.1007/978-3-319-40144-7_8).

### SUMMARY OF INVENTION

In an aspect of the present disclosure, there is provided a method of inducing tissue regeneration, said method comprising administering to said tissue a composition comprising a population of mesenchymal stem cells (MSCs) and a carrier, wherein at least 70% of the MSCs express at least one marker selected from CD73 CD90 or CD105; and less than 5% of MSCs express CD45 marker and HLA-DR marker (Human Leukocyte Antigen-DR isotype); wherein said population of MSCs is a homogeneous population having a size in the range of 15-30 μm; and wherein said composition comprises secretome of said MSCs having VEGF in an amount in the range of 2050 to 2390 μg per million MSCs, and IL-10 in an amount in the range of 1430 to 1690 μg per million MSCs.

In an aspect of the present disclosure, there is provided a method of treating an autoimmune disease in a subject, the method comprising: administering therapeutic amount of a composition comprising a population of mesenchymal stem cells (MSCs) and a carrier to the subject by intra-plantar route, intravenous route, intramuscular route, intraarticular route, intraosseous route, or subcutaneous route; wherein at least 70% of the MSCs express at least one marker selected from CD73 CD90 or CD105; and less than 5% of MSCs express CD45 marker and (Human Leukocyte Antigen-DR isotype) HLA-DR marker; wherein said population of MSCs is a homogeneous population having a size in the range of 15-30 μm; and wherein said composition comprises secretome of said MSCs having VEGF in an amount in the range of 2050 to 2390 μg per million MSCs, and IL-10 in an amount in the range of 1430 to 1690 μg per million MSCs.

These and other features, aspects, and advantages of the present subject matter will be better understood with reference to the following description and appended claims. This summary is provided to introduce a selection of concepts in a simplified form. This summary is not intended to identify key features or essential features of the claimed subject matter, nor is it intended to be used to limit the scope of the claimed subject matter.

### BRIEF DESCRIPTION OF ACCOMPANYING DRAWINGS

The following drawings form a part of the present specification and are included to further illustrate aspects of the present disclosure. The disclosure may be better understood by reference to the drawings in combination with the detailed description of the specific embodiments presented herein.

FIG. 1 depicts the images of the joints of mice model with a) mild arthritis b) moderate arthritis, and c) severe arthritis, and d) recovered animal after treatment, in accordance with the embodiments herein.

FIG. 2 depicts the microscopic observations of a) the articular joints of the Group 1-negative control group animals (Not induced with arthritis), wherein arrows show intact articular cartilage with normal joint cavity and synovial tissue, H&E 10X; and b) minimal cellular infiltration (indicated by A) and bone damage (osteoclastic activity-indicated by B) in Group 6-positive control group animals H&E 10X, in accordance with the embodiments herein.

FIG. 3 depicts the microscopic observations of a) mild hypocellularity (arrow A), mitotic figures indicated by arrow B in the joints of animals from G2 mild subgroup H&E 10X; b) minimal destruction in cartilaginous layer of synovial epithelium (arrow A) with mild mitotic figures (arrow B) of animals from G2 moderate subgroup H&E 10X; and c) mild cellular infiltration (B) and erosion (A) in synovial tissue from Group 2—(Test Product—1 hUCT-MSCs) A1—Intra-planter—1.5×10⁶ cells/ml—H&E 10X, in accordance with the embodiments herein.

FIG. 4 depicts the microscopic observations of a) intact articular cartilage with normal joint cavity and synovial tissue from Group 3—(Test Product—1 hUCT-MSCs) B1—Intravenous—4×10⁶ cells/ml. H&E 10X; and b) minimal destruction in the cartilaginous and bone layer (A). Mitotic figures observed (B) from Group 3—(Test Product—1 hUCT-MSCs) B1—Intravenous-4×10⁶ cells/ml, H&E 10X, in accordance with the embodiments herein.

FIG. 5 depicts the microscopic observations of a) recovery observed in intact articular cartilage with normal joint cavity and synovial tissue with minimal mitotic figures from Group G5—(Test Product—2 hBM-MSCs) B2—Intravenous—4×10⁶ cells/ml-H&E 10X; b) intact articular cartilage with normal joint cavity and synovial tissue from Group 5—(Test Product—2 hBM-MSCs) B2—Intravenous—4×10⁶ cells/ml, H&E 10X; and c) intact articular cartilage with normal joint cavity and synovial tissue from Group 5—(Test Product—2 hBM-MSCs) B2—Intravenous—4×10⁶ cells/ml, H&E 10X, in accordance with the embodiments herein.

FIG. 6 depicts the graphical representation of the concentration of TNFα biomarker in a) mild Group on day 30 (post-transplant); b) moderate Group on day 30 (post-transplant) i.e. first end point and Day 60 (post-transplant) i.e. second end point; and c) severe group on day 30 (post-transplant) i.e. first end point and Day 70 (post-transplant) i.e. second end point, in accordance with the embodiments herein.

FIG. 7 depicts the graphical representation of the concentration IL-10 biomarker in a) mild group on day 30 (post-transplant); b) moderate group on day 30 (post-transplant) i.e. first end point and Day 60 (post-transplant) i.e. second end point, c) severe group on day 30 (post-transplant) i.e. first end point and Day 70 (post-transplant) i.e. second end point, in accordance with the embodiments herein.

### DETAILED DESCRIPTION OF THE INVENTION

Those skilled in the art will be aware that the present disclosure is subject to variations and modifications other than those specifically described. It is to be understood that the present disclosure includes all such variations and modifications. The disclosure also includes all such steps, features, compositions, and compounds referred to or indicated in this specification, individually or collectively, and any and all combinations of any or more of such steps or features.

### Definitions

For convenience, before further description of the present disclosure, certain terms employed in the specification, and examples are delineated here. These definitions should be read in the light of the remainder of the disclosure and understood as by a person of skill in the art. The terms used herein have the meanings recognized and known to those of skill in the art, however, for convenience and completeness, particular terms and their meanings are set forth below.

The articles “a”, “an” and “the” are used to refer to one or to more than one (i.e., to at least one) of the grammatical object of the article.

The terms “comprise” and “comprising” are used in the inclusive, open sense, meaning that additional elements may be included. It is not intended to be construed as “consists of only”.

Throughout this specification, unless the context requires otherwise the word “comprise”, and variations such as “comprises” and “comprising”, will be understood to imply the inclusion of a stated element or step or group of elements or steps but not the exclusion of any other element or step or group of elements or steps.

The term “including” is used to mean “including but not limited to”. “Including” and “including but not limited to” are used interchangeably.

The term “subject”, as used herein, refers to mammals, e.g., human, and non-human mammals. Examples of non-human animals include non-human primates, dogs, cats, cattle, horses, sheep, pigs, goats, rabbits, mice, rats, hamsters, guinea pigs etc. Unless otherwise noted, the terms “patient” or “subject” are used herein interchangeably. Preferably, the subject is human.

The term “therapeutically effective amount” as used herein refers to the amount of composition sufficient to prevent onset or progression of a disease, alleviates or eliminates disease symptoms, promotes disease regression, promotes an increase in frequency and duration of disease symptom-free periods, or prevents impairment or disability due to disease affliction. The ability of a therapeutic composition or agent to promote disease regression can be evaluated using a variety of methods known to the skilled practitioner, such as in human subjects during clinical trials, in animal model systems predictive of efficacy in humans, or by assaying the activity of the composition or agent in in vitro assays.

The term “administering” as used herein refers to the physical introduction of a composition to a subject, using any of the various methods and delivery systems known to those skilled in the art. Administering of the composition may be through intra-plantar route, intravenous route, intramuscular route, intraarticular route, intraosseous route, or subcutaneous route.

Embodiments herein provide a method of inducing tissue degeneration and a method of treating autoimmune disease, in a subject, using a composition comprising MSCs.

Mesenchymal stem cells (MSCs) are mesoderm-derived cells that reside in the stroma of solid organs and function as precursors of non-hematopoietic connective tissues. They can exert profound immunosuppression by modulating T (thymus) and B (bone marrow) cells derived proliferation and differentiation, dendritic cell maturation and natural killer (NK) Cells activity. These immunoregulatory properties encourage a possible use of these cells to modulate autoimmune responses and in the treatment of autoimmune diseases. However, there is still a need for developing a suitable treatment regime using formulations/compositions comprising MSCs.

Accordingly, embodiments herein provide a method of inducing tissue regeneration, and a method of treating an autoimmune disease, in a subject in need thereof, using a composition comprising a population of MSCs and a carrier.

The term “mesenchymal stem cell” or “MSCs”, as used herein, refers to cell population of multipotent cells. According to the International Society for Cellular Therapy (ISCT), the criteria to define MSCs are their plastic-adherent ability; the high expression of positive markers, such as CD105, CD73, CD90, as well as the lack of expression of negative markers, such as CD45, CD34, CD14/CD11b, CD79a/CD19, HLA class II. The MSCs, according to embodiments herein, are characterized by the expression of one or more cell surface markers selected from CD73, CD90, or CD105. In further embodiments herein, the MSCs may be characterized by lack of expression of one or more of the markers selected from HLADR, CD34, or CD45.

In an embodiment, at least 70% of the MSCs express at least one marker selected from CD73 CD90 or CD105. In another embodiment, at least 70%, at least 75%, at least 80%, at least 85%, at least 90% or at least 95% of the MSCs express at least one marker selected from CD73, CD90 or CD105.

In an embodiment, less than 5% of the the MSCs express CD 45 marker and HLADR (Human Leukocyte Antigen—DR isotype) marker. In another embodiment, less than 5%, less than 4%, less than 3%, less than 2% or less than 1%, of the MSCs express CD 45 marker and HLADR marker.

The MSCs may be obtained from commercial sources or, alternatively, derived from tissues of mammals, in particular humans (healthy donors).

In an embodiment, the MSCs are derived from umbilical cord tissue, cord blood, adipose tissue, bone marrow, or dental pulp, preferably the MSCs are derived from umbilical cord tissue (UCT) or bone marrow.

The MSCs isolated from UCT, or bone marrow may be a heterogeneous population of MSCs. The term “heterogeneous population”, as used herein, refers to a mixed population of small, medium, or large sized MSCs. According to embodiments herein, the MSCs having medium size are particularly expanded and produced using the methods well known in the art.

In an exemplary embodiment, the homogeneous population of MSCs is obtained by a process comprising the steps of:
a) culturing a heterogeneous population of MSCs in a culture medium for a period in a range of 20 to 35 days; b) staining the MSCs to obtain a heterogeneous population of stained MSCs; c) adding droplet encapsulation media, comprising a density gradient solution to the heterogeneous population of stained MSCs, to obtain a population of microfluidic droplets, wherein each droplet preferably comprises a single MSC; d) providing the population of microfluidic droplets obtained from step (c) to a microfluidics device; and e) identifying and selecting a homogeneous population of MSCs of medium size in the range of 15 to 30 μm; wherein the step (e) further comprises: imaging the MSCs in the population of microfluidic droplets to record the size of the MSCs; and subjecting the population of microfluidic droplets to automated size-based sorting to obtain a population of homogenous MSCs having medium size; and wherein the step (a) further comprises: reseeding the heterogeneous population of MSCs at a cell density in the range of 5000 to 11,000/cm² and expanding the heterogeneous population of MSCs up to passage 3, wherein the population of MSCs obtained from step (e) are viable MSCs, expressing MSC specific markers selected from CD 73, CD 90, and combinations thereof; and exhibiting increased expression of genes selected from the group consisting of COL12 A1 gene, IGFBP5 gene, THBS2 gene, GREM1 gene, CDH2 gene and combinations thereof. In an embodiment, the homogeneous population of MSCs is of medium size in the range of 15 to 30 μm. In another embodiment, the population of MSCs is of medium size in the range of 17 to 22 μm.

In an embodiment, the composition comprises secretome of said MSCs.

The term “secretome” as used herein refers to the biologically active substances that are secreted by the MSCs and have the paracrine ability. The secretome is composed of cytokines, chemokines, growth factors, proteins, and extracellular vesicles. In an embodiment, the composition comprises secretome of said MSCs comprising VEGF and IL-10.

The term “Vascular endothelial growth factor” or “VEGF” as used herein refers to a family of polypeptides that includes VEGF-A, VEGF-B, VEGF-C, VEGF-D and placental growth factor (PIGF). VEGF shows paracrine and autocrine properties and can act intracellularly, secreting to the extracellular space, participating in the regulation of the cell-cycle and metabolism of cells. VEGF, an important angiogenic factor secreted by MSCs, promotes cell survival by inducing the expression of anti-apoptotic molecules such as Bcl-2.

The term “Interleukin-10” or “IL-10” as used herein refers to a type of cytokine that is found in the secretome of MSCs. IL-10 is characterized by its anti-inflammatory effect related to the induction of immune tolerance. It is an anti-inflammatory cytokine that inhibits the IL-2 and Interferon (IFN)-G. IL-10 act as an inducer for immune tolerance on the dendritic cells. It has been established that IL-10 suppresses the functions of macrophages and neutrophils, inhibits the Th1 immune response, influences NF-kB synthesis and causes expression of anti-inflammatory molecules, such as protease inhibitors and IL-1 and TNFα antagonists.

The major function of IL-10 in induction of immune tolerance is its effect on the antigen presenting cells, particularly on the dendritic cells (DCs). IL-10 suppresses the secretion of pro-inflammatory cytokines (TNFα, IL-1, IL-6, IL-8, IL-12) by DCs and the expression of MHC II molecules, as well as co-stimulatory complex B7 on their surface. In parallel, IL-10 is capable of inducing anergy of T lymphocytes by directly inhibiting the phosphorylation of CD28. Thereby, one of the basic immunosuppressive mechanisms is executed by IL-10 by inducing a tolerogenic type of dendritic cells with reduced HLA-II and B7 expression and by suppression of CD28 (the partner of B7) expression on the surface of the T lymphocytes. IL-10 plays a key role in the development and function of regulatory T cells (Tregs), a specialized subset of T cells that suppress immune responses. By promoting Tregs, IL-10 indirectly contributes to the overall suppression of T cell activation. This “two sided” suppression executed by Il-10 is due to the combined effect resulting from downregulation of co-stimulatory molecules on antigen presenting cells (APCs) along with upregulation of Tregs. Overall, IL-10 acts as a key immunoregulatory molecule, playing a crucial role in this “two-sided” suppression, leading to T cell anergy and preventing excessive immune responses that could damage healthy tissues.

In an embodiment, the composition comprises secretome of said MSCs having VEGF in an amount in the range of 2050 to 2390 μg per million MSCs, and IL-10 in an amount in the range of 1430 to 1690 μg per million MSCs.

In an embodiment, the homogeneous population of MSCs are present in a suspension comprising an excipient selected from DMEM, human serum albumin (HSA) or combinations thereof; preferably the excipient is DMEM and 20% HSA solution in a weight ratio of 1:1.

In an embodiment, the MSCs are autogenic or allogenic to the subject.

The term “carrier” as used herein refers to any known carrier, or adjuvants known to a person skilled in the art, and which can be used for preparing the formulation comprising the population of MSCs for therapeutic applications and is pharmaceutically acceptable.

In an embodiment, the carrier is selected from serelaxin, Ringer's lactate solution, human serum albumin (HSA), DMEM medium, saline solution, phosphate buffered saine (PBS) buffer, Hank's balanced salt solution (HBSS), human plasma, plasma lysate, human albumin, or mixtures thereof, preferably the carrier comprises of serelaxin, Ringer's lactate solution and human serum albumin (HSA), heparin, dextran 40, and hyaluronidase.

It will be apparent to one skilled in the art that the method for preparation of the composition of the present disclosure is not limiting, and that compositions of the invention prepared in any way are included within the scope of the invention. In an embodiment, the composition is obtained by mixing predetermined amounts of the MSCs and the carrier.

In an embodiment, the composition has a pH in the range of 6.4 to 6.6.

In an embodiment, the composition has an osmolarity in the range of 270 to 310 mOsm/L.

The compositions of the present disclosure may include, in addition to MSCs, non-cellular components. Examples of such non-cellular components include but are not limited to cell culture media, which may comprise one or more of proteins, amino acids, nucleic acids, nucleotides, co-enzyme, antioxidants and metals.

Embodiments herein provide a method of inducing tissue regeneration.

In embodiment, there is provided a method of inducing tissue regeneration, comprising administering to said tissue a composition comprising a population of mesenchymal stem cells (MSCs) and a carrier, wherein at least 70% of the MSCs express at least one marker selected from CD73 CD90 or CD105; and less than 5% of MSCs express CD45 marker and HLA-DR marker (Human Leukocyte Antigen—DR isotype); wherein said population of MSCs is a homogenous population having a size in the range of 15-30 μm; wherein said population of MSCs is a homogeneous population having a size in the range of 15-30 μm; and wherein said composition comprises secretome of said MSCs having VEGF in an amount in the range of 2050 to 2390 μg per million MSCs, and IL-10 in an amount in the range of 1430 to 1690 μg per million MSCs.

In an embodiment, said tissue is selected from epithelial tissue; connective tissue like bone tissue, cartilage tissue or elastic tissue; muscle tissue, or nervous tissue.

Embodiments herein include a method of treating an autoimmune disease in a subject.

In an embodiment, there is provided a method of treating an autoimmune disease in a subject in need thereof, the method comprising: administering a therapeutically effective amount of a composition comprising a population of mesenchymal stem cells (MSCs) and a carrier to the subject by intraplantar route, intravenous route, intramuscular route, intraarticular route, intraosseous route, or subcutaneous route; wherein at least 70% of the MSCs express at least one marker selected from CD73 CD90 or CD105; and less than 5% of MSCs express CD45 marker and (Human Leukocyte Antigen—DR isotype) HLA-DR marker; wherein said population of MSCs is a homogenous population having a size in the range of 15-30 μm; and wherein said composition comprises secretome of said MSCs having VEGF in an amount in the range of 2050 to 2390 μg per million MSCs, and IL-10 in an amount in the range of 1430 to 1690 μg per million MSCs.

In an embodiment, the auto immune disease is selected from the group of consisting of Acromegaly, Acquired Aplastic Anemia, Acquired Hemophilia, Agammaglobulinemia, Alopecia Areata, Ankylosing Spondylitis (AS), Anti-NMDA Receptor Encephalitis, Antiphospholipid Syndrome (APS), Arteriosclerosis, Autoimmune Addison's Disease (AAD), Autoimmune Autonomic Ganglionopathy (AAG), Autoimmune Encephalitis (AE)/Acute Disseminated Encephalomyelitis (ADEM), Autoimmune Gastritis, Autoimmune Hemolytic Anemia (AIHA), Autoimmune Hepatitis, Autoimmune Hyperlipidemia, Autoimmune Hypophysitis/Lymphocytic Hypophysitis, Autoimmune Inner Ear Disease (AIED), Autoimmune Lymphoproliferative Syndrome (ALPS), Autoimmune Myelofibrosis (AIMF), Autoimmune Myocarditis, Autoimmune Oophoritis, Autoimmune Pancreatitis (AIP), Autoimmune Polyglandular Syndromes (APS), Autoimmune Progesterone Dermatitis (APD), Autoimmune Retinopathy (AIR), Autoimmune Sudden Sensorineural Hearing Loss, Balo Disease/Concentric Sclerosis, Behçet's Disease, Birdshot Chorioretinopathy/Birdshot Uveitis, Bullous Pemphigoid, Castleman Disease, Celiac Disease, Chagas Disease, Chronic Inflammatory Demyelinating Polyneuropathy (CIDP), Chronic Autoimmune Urticaria, Churg-Strauss Syndrome/Eosinophilic Granulomatosis with Polyangiitis (EGPA), Cogan's Syndrome (CS), Cold Agglutinin Disease (CAD), Crest Syndrome, Crohn's Disease, Stricturing Crohn's Disease, Cronkhite-Canada Syndrome (CCS), Cryptogenic Organizing Pneumonia (COP), Dermatitis Herpetiformis (DH), Dermatomyositis, Diabetes, Type 1 (TID), Discoid Lupus Erythematosus (DLE), Dressler's Syndrome/Post myocardial Infarction/Post pericardiotomy Syndrome, Eczema/Atopic Dermatitis, Eosinophilic Fasciitis, Erythema Nodosum, Essential Mixed Cryoglobulinemia, Evans Syndrome, Fibrosing Alveolitis/Idiopathic Pulmonary Fibrosis (IPF), Giant Cell Arteritis/Temporal Arteritis/Horton's Disease, Giant Cell Myocarditis, Glomerulonephritis (GN), Goodpasture's Syndrome/Anti-Gbm/Anti-Tbm Disease, Granulomatosis With Polyangiitis (GPA)/Wegener's Granulomatosis, Graves' Disease (GD), Guillain-Barre Syndrome (GBS), Hashimoto's Thyroiditis/Autoimmune Thyroiditis, Henoch-Schölein Purpura (HSP)/Iga Vasculitis, Hidradenitis Suppurativa, Hurst's Disease/Acute Hemorrhagic Leukoencephalitis (AHLE), Hypogammaglobulinemia, Iga Nephropathy/Berger's Disease, Immune-Mediated Necrotizing Myopathy (IMNM), Immune Thrombocytopenia (Itp)/Autoimmune Thrombocytopenia Purpura, Inclusion Body Myositis (IBM), Igg4-Related Sclerosing Disease (ISD), Interstitial Cystitis, Juvenile Idiopathic Arthritis (Jia)/Adult-Onset Still's Disease, Juvenile polymyositis/Juvenile dermatomyositis/juvenile myositis, Kawasaki disease, Lambert-Eaton Myasthenic Syndrome (LEMS), Leukocytoclastic vasculitis, Lichen Planus, Lichen Sclerosus, Ligneous conjunctivitis, Linear Iga Disease (LAD), Lupus Nephritis (LN), Lyme Disease/Chronic Lyme Disease/Post-Treatment Lyme Disease Syndrome (PTLDS), Lymphocytic colitis/microscopic colitis, Lymphocytic hypophystitis/autoimmune hypophystitis, Ménière's Disease, Microscopic Polyangiitis (MPA)/ANCA-Associated Vasculitis, Mixed Connective Tissue Disease (MCTD), Mooren's ulcer, Mucha-Habermann disease, Multifocal motor neuropathy, Multiple Sclerosis (MS), Myalgic Encephalomyelitis/Chronic Fatigue Syndrome (ME/CFS), Myasthenia Gravis (MG), Narcolepsy, Neuromyelitis Optica/Devic's Disease, Ocular Cicatricial Pemphigoid, Opsoclonus-myoclonus syndrome (OMS), Palindromic Rheumatism, Paraneoplastic Cerebellar Degeneration (PCD), Paraneoplastic Pemphigus, Parry-Romberg Syndrome (PRS)/Hemifacial Atrophy (HFA)/Progressive Facial Hemiatrophy, Paroxysmal Nocturnal Hemoglobinuria (PNH), Peripheral uveitis/pars planitis, PANS/PANDAS, Parsonage-Turner Syndrome (PTS), Pemphigoid Gestationis (PG), Pemphigus Foliaceus, Pemphigus Vulgaris, Pernicious anemia, POEMS Syndrome, Polyarteritis Nodosa (PAN), Polymyalgia Rheumatica, Polymyositis, Postural Orthostatic Tachycardia Syndrome (Pots), Primary Biliary Cirrhosis (PBC), Primary Sclerosing Cholangitis (PSC), Psoriasis, Palmoplantar Pustulosis (PPP), Psoriatic Arthritis, Pulmonary fibrosis, idiopathic (IPF), Pure Red Cell Aplasia (PRCA), Pyoderma gangrenosum, Rasmussen's encephalitis, Raynaud's Syndrome, Reactive Arthritis, Reflex sympathetic dystrophy syndrome (RSD)/Complex regional pain syndrome (CRPS), Relapsing Polychondritis (RP), Restless leg syndrome (RLS)/Willis-Ekbom disease, Rheumatic Fever, Rheumatoid Arthritis (RA), Sarcoidosis, Schmidt Syndrome/Autoimmune Polyendocrine Syndrome Type II, Scleritis, Scleroderma, Sclerosing Mesenteritis/Mesenteric Panniculitis, Serpiginous choroidopathy, Sjögren's Syndrome, Stiff person syndrome (SPS), Small Fiber Sensory Neuropathy (SFSN), Small Fiber Sensory Neuropathy (SFSN), Systemic Lupus Erythematosus (SLE), Subacute bacterial endocarditis (SBE), Subacute cutaneous lupus, Susac's syndrome, Sydenham's Chorea, Sympathetic ophthalmia, Takayasu's arteritis (vasculitis), Testicular Autoimmunity, Tolosa-Hunt syndrome, Transverse myelitis (TM), Tubulointerstitial nephritis uveitis syndrome (TINU), Ulcerative Colitis, Undifferentiated Connective Tissue Disease, Uveitis, Vasculitis, VEXAS Syndrome, Vogt-Koyanagi-Harada syndrome (VKH), Osteoarthritis, AVN, vertebral compression factor, urethral stricture, ureteric stricture, eye fibrosis, heart fibrosis, hepatic fibrosis, intestinal fibrosis, lung fibrosis, Pancreas fibrosis, renal fibrosis, and skin fibrosis. In another embodiment, the disease is mild auto immune disease. In one another embodiment, the disease is moderate auto immune disease. In yet another embodiment, the disease is severe auto immune disease.

In an embodiment, the auto immune disease is arthritis. In another embodiment, the autoimmune disease is mild arthritis, moderate arthritis, or severe arthritis. The diagnosis and severity classification of arthritis are preferably carried out using clinically accepted criteria, such as those established by The European League Against Rheumatism (EU LAR) e.g. Disease Activity Score 28-joint count (DAS28) or using an inhouse disease activity disease measurement protocol.

In an embodiment, the mild autoimmune disease is mild arthritis with an arthritis scoring in the range of 1≥ to ≤2, and/or a Disease Activity Score in 28 joints (DAS 28 score) in the range of 2.6≥ to ≤3.1

In an embodiment, the moderate autoimmune disease is moderate arthritis with an arthritis scoring in the range of 2≥ to ≤3, and/or a DAS 28 score in the range of 3.1≥ to ≤5.1

In yet another embodiment, the severe autoimmune disease is severe arthritis with an arthritis scoring in the range of 3≥ to ≤4, and/or a DAS 28 score>5.1.

In an embodiment, the mild autoimmune disease is mild arthritis with an arthritis scoring in the range of 1≥ to ≤2, and/or a Disease Activity Score in 28 joints (DAS 28 score) in the range of 2.6≥ to ≤3.1; wherein the moderate autoimmune disease is moderate arthritis with an arthritis scoring in the range of 2> to ≤3, and/or a DAS 28 score in the range of 3.1≥ to ≤5.1; and wherein the severe autoimmune disease is severe arthritis with an arthritis scoring in the range of 3≥ to ≤4, and/or a DAS 28 score>5.1.

The dosage of the composition comprising MSCs will vary depending on the symptoms, age and body weight of the subject, the nature and severity of the disease to be treated, the route of administration. The compositions of the invention may be administered in a single dose or in divided doses. Appropriate dosages for MSCs may be determined by known techniques.

In an embodiment, the composition is administered in a range of 1 to 5 doses at a time interval of 1 to 2 months; wherein each dose comprises of 0.5 to 10 million cells per kg of the subject's body weight. In further embodiments, the dose may comprise 1 million cells, 2 million cells, 3 million cells, 4 million cells, 5 million cells, 6 million cells, 7 million cells, 8 million cells, 9 million cells, or 10 million cell per kg of the subject's weight.

In an alternative embodiment, the composition may be administered to the subject as a fixed dose, independent of subject's weight. Typically said dose is between about 10 million cells and 500 million cells, e.g. said dose is about 10×10⁶ cells, 50×10⁶ cells, 10×10⁷ cells, or 50×10⁷ cells.

The treatment outcome post administering the composition may be monitored by assessing the level of biomarkers in the subject. Examples of biomarkers include but are not limited serum biomarkers, such as IL-10 and TNF-α. Assessing the levels of such biomarkers are well known in art. According to embodiments herein, the serum levels of IL-10 and TNF-α may be assessed using ELISA (enzyme linked immunosorbent assay).

In an embodiment, the method increases the levels of IL-10 in the subject; wherein the increase in levels of IL-10 estimated 30 days post administration of the composition in the subject having mild autoimmune disease is in the range of 30 to 50% as compared to a control; wherein the increase in levels of IL-10 estimated 30 days post administration of the composition in the subject having moderate autoimmune disease is in the range of 40 to 60% as compared to a control; and wherein the increase in levels of IL-10 estimated 30 days post administration of the composition in the subject having severe autoimmune disease is in the range of 70 to 90% as compared to a control.

In an embodiment, the method increases the levels of IL-10 in the subject; wherein the increase in levels of IL-10 estimated 60 days post administration of the composition in the subject having moderate autoimmune disease is in the range of 90 to 120% as compared to a control; and wherein the increase in levels of IL-10 estimated 70 days post administration of the composition in the subject having severe autoimmune disease is in the range of 80 to 100% as compared to a control.

In an embodiment, the method decreases the levels of TNF-α in the subject; wherein the decrease in levels of TNF-α estimated in the subject having mild autoimmune disease 30 days post administration of the composition is in the range of 30 to 50% as compared to a control; wherein the decrease in levels of TNF-α estimated in the subject having moderate autoimmune disease 30 days post administration of the composition is in the range of 50 to 70% as compared to a control; and wherein the decrease in levels of TNF-α estimated in the subject having severe autoimmune disease 30 days post administration of the composition is in the range of 60 to 80% as compared to a control.

In an embodiment, the method decreases the levels of TNF-α in the subject; wherein the decrease in levels of TNF-α estimated in the subject having moderate autoimmune disease 60 days post administration of the composition is in the range of 80 to 100% as compared to a control; and wherein the decrease in levels of TNF-α estimated in the subject having severe autoimmune disease 70 days post administration of the composition is in the range of 80 to 100% as compared to a control.

In an embodiment, said subject having mild autoimmune disease exhibits 95 to 100% recovery of symptoms 30 days post administration of the composition; wherein said subject having moderate autoimmune disease exhibits 55 to 65% recovery of symptoms 60 days post administration of the composition; and wherein said subject having severe autoimmune disease exhibits 35 to 45% recovery of symptoms 60 days post administration of the composition.

Although the subject matter has been described with reference to specific embodiments, this description is not meant to be construed in a limiting sense. Various modifications of the disclosed embodiments, as well as alternate embodiments of the subject matter, will become apparent to persons skilled in the art upon reference to the description of the subject matter. It is therefore contemplated that such modifications can be made without departing from the spirit or scope of the present subject matter as defined.

### LIST OF ABBREVIATIONS

° C. Degree Celsius
% Percentage
μL Microliter
CFA Complete Freund's Adjuvant
CPCSEA Committee for the Purpose of Control and Supervision of Experiments on Animals
ELISA Enzyme Linked Immunosorbent Assay
g Gram
Hrs Hours
hUCT-MSCs Human Umbilical Cord Tissue Mesenchymal Stem Cells
hBM-MSC Human bone marrow Mesenchymal Stem Cells
IAEC Institutional Animal Ethics Committee
IL-10 Interleukin 10 Cytokine
IEC Institutional Ethics Committee
IC-SCR Institutional Committee for Stem Cells Research
Kg Kilogram
Ltd Limited
MI Milliliter
min Minute
MSC Mesenchymal Stem Cells
N Number of Animals
Pvt Private
SD Standard Deviation
SOP Standard Operating Procedure
TNF-α Alpha Tumor necrosis Factor
CFA Complete Freund's Adjuvant
H Hematoxylin
E Eosin
IV Intravenous
IP Intra-planter
COA Certificate of Analysis

### EXAMPLES

The disclosure will now be illustrated with working examples, which is intended to illustrate the working of disclosure and not intended to take restrictively to imply any limitations on the scope of the present disclosure. Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices, and materials are described herein. It is to be understood that this disclosure is not limited to particular methods, and experimental conditions described, as such methods and conditions may vary.

### Materials

### Test Products (Composition Used) Details

| Components | Test Product 1 | Test Product 2 |
|---|---|---|
| MSCs | Medium sized MSCs | Medium sized MSCs |
| | derived from hUCT- | derived from hBM- |
| | MSCs | MSCs |
| Excipient | DMEM and 20% HSA | DMEM and 20% HSA |
| | solution (1:1) | solution (1:1) |
| Carrier | RL with 1% HSA, 1 | RL with 1% HSA, 1 |
| | ng/ml serelaxin, 1% | ng/ml serelaxin, 1% |
| | dextran 40 and heparin | dextran 40 and heparin |
| | 1000 U/ml | 1000 U/ml |
| pH | 6.5 | 6.5 |
| Osmolality | 273 mOsm/L | 273 mOsm/L |

### Example 1: Determination of Efficacy and Safety of Composition Comprising MSCs in CFA Induced Arthritis Model

All procedures to be followed for the conduct of the animal study were in accordance with the Standard Operating Procedures and the guidelines set by the Committee for the Purpose of Control and Supervision of Experiments on Animals (CPCSEA) as published in The Gazette of India, Dec. 15, 1998. A total of 75 male Sprague Dawley Rats (procured from Global Bioresearch Solution Pvt. Ltd.) were acclimatized for a minimum period of nine days prior to administration of composition comprising MSCs. During this period, all animals were observed once a day for its clinical signs and twice a day for its mortality.

The acclimatized animals were divided into 7 different groups, including G1-control, G2-1A1, G3-2A2, G4-1B1, G5-2B2, G6-positive control and G7-reference standard (Table 1).

### Development of Arthritis Model

Adjuvant induced arthritis (AIA) was induced by single subcutaneous (SC) injection of 0.1 ml of CFA (Sigma Aldrich; Product code: 1002865531) containing 10 mg/ml of heat killed Mycobacterium tuberculosis in all animals from group G2 to G7. Animals from group G1 received only normal saline solution by intravenous route and were considered as negative control. All animals injected with CFA and monitored for development of arthritis for a period of 10 days.

### Arthritis Scoring or Paw Swelling

Arthritis developed was scored on a scale of 0-4, where 0=no swelling, 1=minimal or slight swelling and erythema of the limb, 2=mild swelling and erythema of the limb, 3=moderate or gross swelling and erythema of the limb and 4=severe swelling. The scoring was performed by visual observations by two different technicians to minimize the error on paw swelling score.

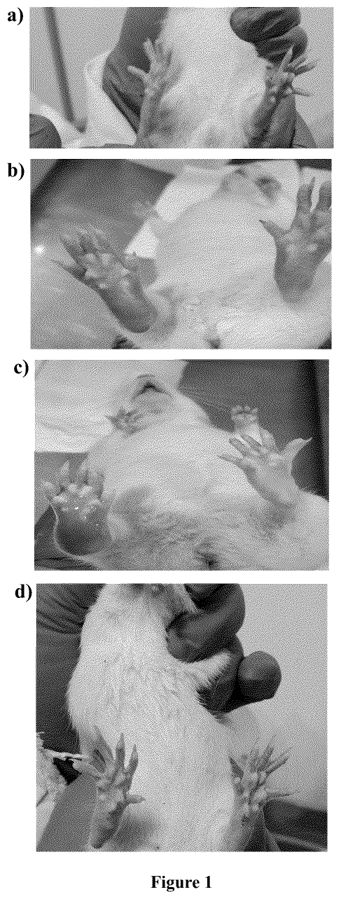

After induction of arthritis, the animals were allocated to different treatment groups randomly. Based on severity score of arthritis, animals were further divided into mild, moderate, and severe subgroups for each group (FIG. 1a-c). For development of severe arthritis, severe group animals were administered with second dose of CFA after 12 days of first CFA dose.

Six animals each from G2, G3, G4 and G5 groups, having arthritis score 2=mild and three animals each from G2, G3, G4 and G5 groups having arthritis score 3=moderate arthritis and score 4=severe, were injected with Test product-1 (composition comprising hUCT-MSCs) A1 via intra-planter at hind paws (paw region) and Test product-1 (hUCT-MSCs) B1 via intravenous route. Similar procedure was followed for Test product-2 (composition comprising hBM-MSC) A2 and Test product-2 (hBM-MSC) B2.

After induction and development of arthritis, all the animals received the treatment of test item on day 1 and day 10. Animals from group G6 did not receive any treatment and were considered as positive control. In case of group G7, animals having arthritis score 2 or 3 received Methotrexate (MTX) orally (p.o.) at 7 mg/kg once a week for 5 weeks in mild and 9 weeks for moderate RA group. Considering the maximum tolerable dose for human of 50 kg body weight, 1×10⁹ (1000 million) this study was designed to inject 4×10⁶ MSCs for the 200-gm rat model for intravenous route of administration and 1.5×10⁶ cells for intraplantar route of administration. During study, after dosing of test products, all the animals were observed for body weight on weekly basis, other treatment related clinical signs daily and morbidity/mortality twice a day. Daily arthritis score was determined, haematology analysis was performed during the sacrifice.

### 1. Efficacy

### Haematology

After completion of observation, period blood samples from all treatment group animals were collected from retro-orbital sinuses for evaluation of haemoglobin content (HGB), total red blood cell count (RBC), % packed cell volume (% PCV), total white blood cells count (WBC) and erythrocyte sedimentation rate (ESR).

### Histopathology

After completion of blood collection, animals were euthanized by CO2 asphyxiation, hind paws were collected in 10% neutral buffered formalin, decalcified, and subjected to routine histopathological examination, stained with Hematoxylin and Eosin stain.

### Biomarkers Analysis

Serum collected from each animal were separated and samples were stored at −20° C. and used for cytokines estimation viz., TNFα (Make: Thermofischer; Catalogue no.: KRC3011), IL-10 (Make: Thermofischer; Catalogue no.: BMS629) by ELISA method as per the standard protocol provided by the Manufacturer.

### 2. Safety Observations

### Mortality and Clinical Signs Observation

After dosing of test products, all the animals were observed carefully for treatment related clinical signs daily and twice for morbidity/mortality.

### Body Weight

Body weights of all the animals were recorded on weekly basis.

### Haematology

After completion of observation period blood samples from all treatment group animals were collected from retro-orbital sinuses for evaluation of haemoglobin content (HGB), total red blood cell count (RBC), % packed cell volume (% PCV), total white blood cells count (WBC) and erythrocyte sedimentation rate (ESR).

### Data Analysis

All the individual animal data were summarized in terms of groups to obtain mean and standard deviation of mean. Data were analyzed using ANNOVA using Graph Pad Prism 7.03 software. All analysis and comparisons were evaluated at the 5% (P<0.05) level in comparison with control.

TABLE 1
Allocation of animals to different treatment groups

| S. | | | Pre- | | | |
|---|---|---|---|---|---|---|
| No. | Group | | treatment | Treatment | Route | Dose* |
| 1 | G1 | | — | Normal | Intra- | 2 ml/kg |
| | (Negative Control) | | | Saline | venous | |
| 2 | G2 | Mild | CFA | Test | Intra- | 1.5 × 10⁶ |
| | (Test Product-1 hUCT- | Moderate | induction | Product- | planter | cells |
| | MSCs) A1 | Severe* | 0.1 | 1A1 | | per 50 μL |
| 3 | G3 | Mild | mL/animal | Test | Intra- | 4 × 10⁶ |
| | (Test Product-1 | Moderate | by | Product- | venous | cells |
| | hUCT-MSCs) B1 | Severe* | Subcutaneous | 1B1 | | per mL |
| 4 | G4 | Mild | route | Test | Intra- | 1.5 × 10⁶ |
| | (Test Product-2 hBM- | Moderate | | Product- | planter | cells |
| | MSC) A2 | Severe* | | 2A2 | | per 50 μL |
| 5 | G5 | Mild | | Test | Intra- | 4 × 10⁶ |
| | (Test Product-2 hBM- | Moderate | | Product- | venous | cells |
| | MSC) B2 | Severe* | | 2B2 | | per mL |
| 6 | G6 | | | Nil | | |
| | (Positive Control) | | | | | |
| 7 | G7(Reference item) # - | Mild | | Methotrexate | Orally | 7 |
| | Methotrexate | Moderate | | | once a | mg/kg |
| | | | | | week | |

### Observation and Results

### 1. Efficacy:

Development of Arthritis Model: The injection of CFA induced inflammation in the paw region of all the animals. Second injection caused severe inflammation in the paw region of all the animals indicated the successful development of arthritis model.

Arthritis Scoring or Paw Swelling: As per Table 2a, Animals from G1 group non-arthritis did not reveal any abnormal signs.

In case of positive control animals receiving only CFA injection from G6 group, 5/10 showed score of 3=moderate and 5/10 animals remained at the score of 4=severe till sacrifice (on day 71).

In case of standard group G7 receiving methotrexate orally, had two subgroups of mild and moderate having 5 animals each. In case of mild subgroup 5/5 showed score of 1=minimal. In case of moderate subgroup ⅘ animals showed score of 1=minimal and ⅕ showed scored of 2-mild till sacrifice (on day 64).

Test Product 1 (hUCT-MSCs): As per details given in table No. 2a, 3/6 animals from G2 group, mild subgroup receiving test product 1-A1 through intra-plantar route completely recovered and remaining 3/6 animals showed score of 1=minimal till sacrifice (on day 34). 6/6 animals from G3 group receiving test product 1-B1 through intra-venous route showed score of 1=minimal till sacrifice (on day 34).

⅔ animals from G2 group, moderate subgroup receiving test product 1-A1 through intra-plantar route showed score of 1=minimal and remaining ⅓ animals showed score of 2=mild till sacrifice (on day 39). ⅓ animals from G3 group receiving test product 1-B1 through intra-venous route showed score of 1=minimal till sacrifice and remaining ⅔ animals showed score of 2=mild till sacrifice (on day 39).

⅔ animals from G2 group, severe subgroup receiving test product 1-A1 through intra-plantar route showed score of 1=minimal and remaining ⅓ animals showed score of 2=mild till sacrifice (on day 45). ⅓ animals from G3 group receiving test product 1-B1 through intra-venous route showed score of 1=minimal till sacrifice and remaining ⅔ animals showed score of 2=mild till sacrifice (on day 45).

TABLE 2a
Arthritis scoring and Paw swelling summary
results of Test Product 1 (hUCT-MSCs)

| | Group | | Test | | | |
|---|---|---|---|---|---|---|
| | of | | Product | | Arthritic | Total |
| Sr. No. | animals | Subgroup | Name | ROA | Score | animal |
| 1 | G2 | Mild | 1-A1 | IP | Complete | 3/6 |
| | | | | | Recovery | |
| 2 | G2 | | 1-A1 | IP | 1 = Minimal | 3/6 |
| | | | | | till Sacrifice | |
| | | | | | (on day 34) | |
| 3 | G3 | | 1-B1 | IV | 1 = Minimal | 6/6 |
| | | | | | till Sacrifice | |
| | | | | | (on day 34) | |
| 4 | G2 | Moderate | 1-A1 | IP | 1 = Minimal | 2/3 |
| | | | | | till Sacrifice | |
| | | | | | (on day 39) | |
| 5 | G2 | | 1-A1 | IP | 2 = Mild | 1/3 |
| | | | | | till Sacrifice | |
| | | | | | (On day 39) | |
| 6 | G3 | | 1-B1 | IV | 1 = | 1/3 |
| | | | | | Minimal till | |
| | | | | | Sacrifice (on | |
| | | | | | day 39) | |
| 7 | G3 | | 1-B1 | IV | 2 = Mild | 2/3 |
| | | | | | till Sacrifice | |
| | | | | | (On day 39) | |
| 8 | G2 | Severe | 1-A1 | IP | 1 = Minimal | 2/3 |
| 9 | G2 | | 1-A1 | IP | 2 = Mild | 1/3 |
| | | | | | till Sacrifice | |
| | | | | | (On day 45) | |
| 10 | G3 | | 1-B1 | IV | 1 = | 1/3 |
| | | | | | Minimal till | |
| | | | | | Sacrifice | |
| 11 | G3 | | 1-B1 | IV | 2 = Mild | 2/3 |
| | | | | | till Sacrifice | |
| | | | | | (On day 45) | |

Test Product 2 (hBM-MSCs): As per details given in Table 2b, ⅙ animals from G4 group, mild subgroup receiving test product 2-A2 through intra-plantar route complete recovered and remaining ⅚ animals showed score of 1=minimal till sacrifice (on day 34). ⅚ animals from G5 group receiving test product 2-B2 through intra-venous route complete recovered and ⅙ showed score of 1=minimal till sacrifice (on day 34).

3/3 animals from G4 group, moderate subgroup receiving test product 2-A2 through intra-plantar showed score of 1=minimal till sacrifice (on day 39). Similarly, 3/3 animals from G5 group, moderate subgroup receiving test product 2-B2 through intra-venous route showed score of 1=minimal till sacrifice (on day 39)

⅔ animals from G4 group, severe subgroup receiving test product 2-A2 through intra-plantar showed score of 1=minimal and remaining ⅓ animals showed score of 2=mild till sacrifice (on day 45). Similarly, 3/3 animals from G5 group, severe subgroup receiving test product 2-B2 through intra-venous route showed score of 1=minimal till sacrifice (on day 45).

TABLE 2b
Arthritis scoring and Paw swelling summary
results of Test Product 2 (hBM-MSCs)

| | Group | | Test | | | |
|---|---|---|---|---|---|---|
| Sr. | of | Sub- | Product | | Arthritic | Total |
| No. | animals | group | Name | ROA | Score | animal |
| 1 | G4 | Mild | 2-A2 | IP | Complete | 1/6 |
| | | | | | Recovery | |
| 2 | G4 | | 2-B2 | IP | 1 = Minimal | 5/6 |
| | | | | | till Sacrifice | |
| | | | | | (on day 34) | |
| 3 | G5 | | 2-B2 | IV | Complete | 5/6 |
| | | | | | Recovery | |
| 4 | G5 | Moderate | 2-B2 | IV | 1 = Minimal | 1/6 |
| | | | | | till Sacrifice | |
| | | | | | (on day 34) | |
| 5 | G4 | | 2-A2 | IP | 1 = Minimal | 3/3 |
| | | | | | till Sacrifice | |
| | | | | | (on day 39) | |
| 6 | G5 | | 2-B2 | IV | 1 = Minimal | 3/3 |
| | | | | | till Sacrifice | |
| | | | | | (on day 39) | |
| 7 | G4 | Severe | 2-A2 | IP | 1 = Minimal | 2/3 |
| 8 | G4 | | 2-A2 | IP | 2 = Mild till | 1/3 |
| | | | | | Sacrifice (On | |
| | | | | | day 45) | |
| 9 | G5 | | 2-B2 | IV | 1 = Minimal | 3/3 |
| | | | | | till Sacrifice | |
| | | | | | (on day 45) | |

TABLE 2c
Arthritis scoring outcome

| Dose severity | Route of administration | Improvement |
|---|---|---|
| Mild | Both IP and IV | 100% recovery in IV and IP |
| Moderete | Both IP and IV | 60% recovery in IV and IP |
| Severe | Both IP and IV | 40% recovery in IV and IP |

Overall, the mean arthritis score was reduced to minimal by both the test products. In mild group, intravenous administration of test product 1 (hUCT MSC) showed 100% reduction of arthritic score (Table 2c). In case of moderate and severe groups test product 2 (hBM MSC) showed better results by intravenous route.

Haematology Analysis: All the haematological parameters including WBC, RBC count, haemoglobin and ESR of all the groups were comparable with control group. Only in group G7 (Reference control), WBC count was significantly higher as compared with G1 group animals.

Histopathology Analysis: Histopathological examination with H and E stain revealed that the damaged caused by CFA treatment was repaired by treatment of both test products groups administered by Intravenous route.

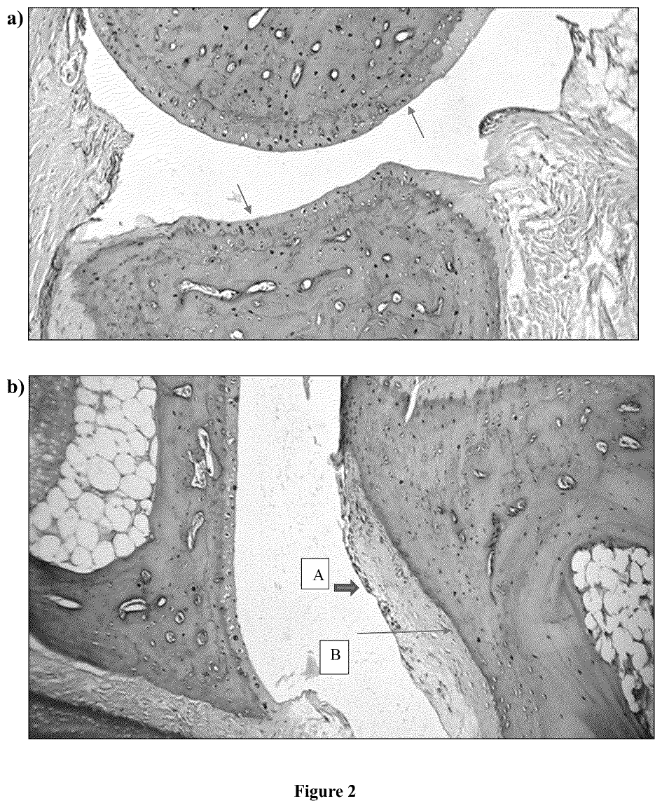

Microscopic observations of the articular joints of the negative control group animals (Not induced with arthritis) revealed normal architecture and nothing abnormal was detected with respect to uniformity of chondrocytic layer, presence of zones of articular cartilage and cell organization in all animals. The uniformity of chondrocyte layer was slightly less uniform in one animal (Refer FIG. 2a)

Out of 3 animals from positive control group (G6) sacrificed along with mild subgroup animals, only one animal showed minimal recovery in terms of uniformity of chondrocyte layer. Other ⅔ animals did not show any signs of recovery.

From reference group (G7) ⅘ animals showed minimal recovery towards uniformity of chondrocyte layer and zone formation.

Positive control group animals (G6) from moderate subgroup did not show recovery except one animal showing minimal cell growth (FIG. 2b).

In case of reference group (G7) moderate subgroup, ⅗ animals showed minimal recovery towards chondrocyte layer and cell regeneration.

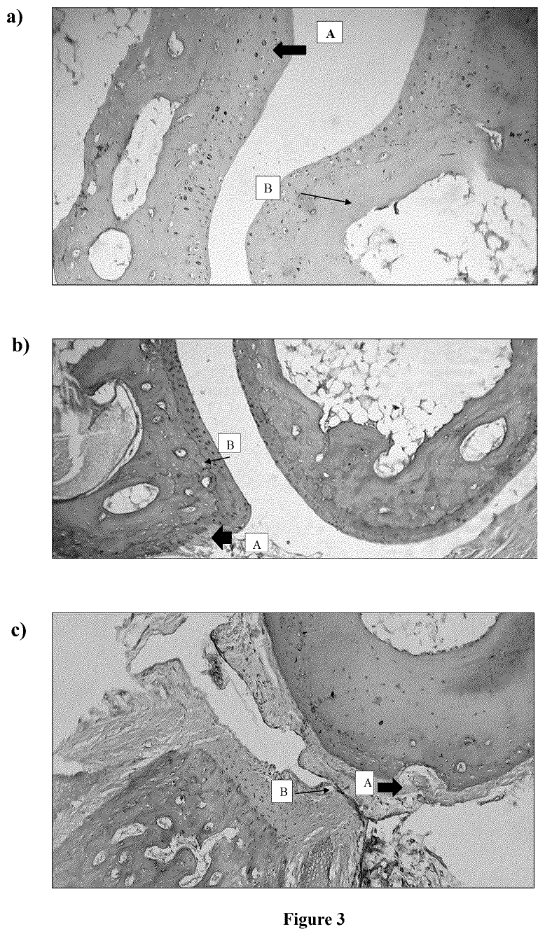

Test Product 1 (hUCT-MSCs): Microscopic examination of the joints of animals from G2 mild subgroup revealed that test product 1-A1 showed reparative changes when administered either by intra-plantar or by intravenous routes. However, the incidence of changes, such as uniformity in the chondrocyte layer, presence of zones of articular cartilage was better in animals receiving test item by intravenous route (6/6) than in animals receiving it via intraplantar route ( 4/6) (FIG. 3).

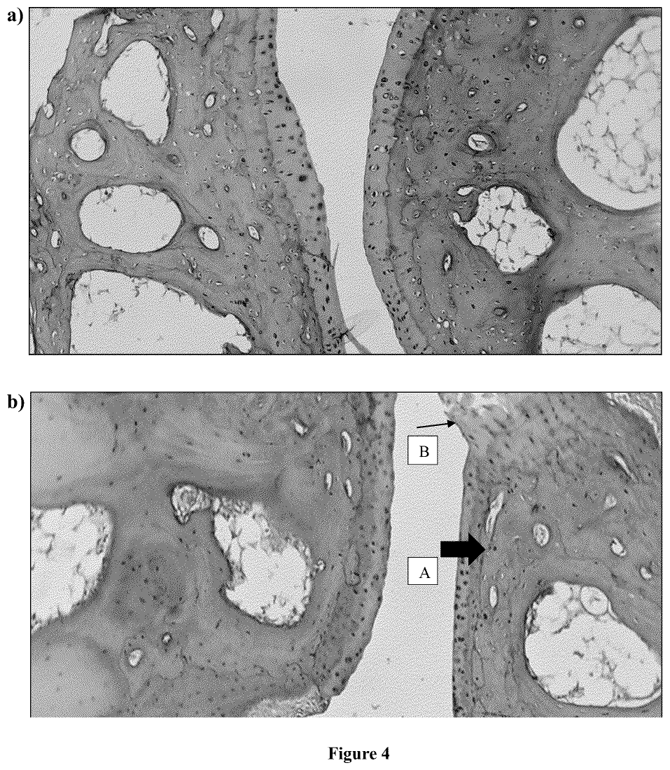

In case of moderate subgroup animals receiving test product 1A1 by intraplantar route, only one animal (⅓) showed some recovery, whereas all 3/3 animals receiving this test product via intravenous route showed recovery (FIG. 4).

All severe group animals receiving test product 1A1 either via intra-plantar or intravenous route showed signs of recovery. However, both routes ⅓ did not have proper zone formation. Test product 2A2 treatment via intra-plantar route showed proper zone formation in ⅓ animals, and treatment via intravenous route showed proper zone formation in ⅔ animals. There were no animals in other groups in severe subgroup.

Test Product 2 (hBM-MSCs): Test product 2-A2 showed reparative changes when administered either by intra-plantar or by intravenous routes. However, the incidence of changes such as uniformity in the chondrocyte layer, presence of zones of articular cartilage was better in animals receiving test item by intravenous route (⅚) than in animals receiving it via intra-plantar route ( 3/6).

All (3/3) the animals from moderate subgroup receiving test product 2A2 either via intra-plantar route (G4) or via intravenous route (G5) showed recovery, however signs were better in the animals receiving the test item via intravenous route.

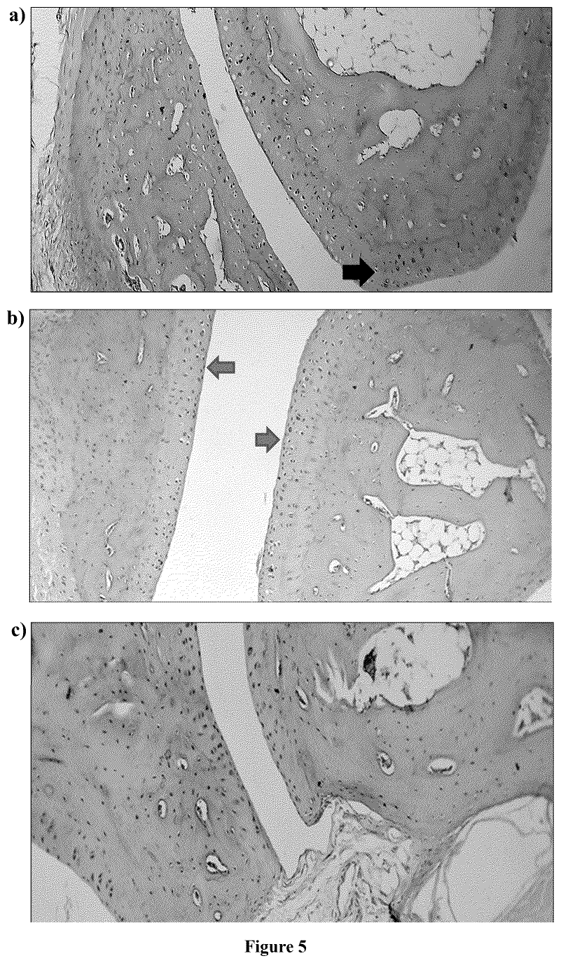

All severe group animals receiving test product 1A1 either via intra-plantar or intravenous route showed signs of recovery. However, both routes ⅓ did not have proper zone formation. Test product 2A2 treatment via intra-plantar route showed proper zone formation in ⅓ animals, and treatment via intravenous route showed proper zone formation in ⅔ animals. There were no animals in other groups in severe subgroup (FIG. 5).

Biomarker Analysis: Biomarker analysis is the most crucial end point since all international studies are heavily focused on this parameter of improvement. The TNF-α and IL-10 level changes in treated groups as compared with those of positive control and reference groups. Biomarker Analysis of the serum sample collected from the mice shows following observations.

### Detection of TNF-α Level in Blood Serum

In case of mild group, both products 1 and 2 are equally effective in treating the mild RA using both intravenous and intra plantar route (Table 3a and Table 3b;

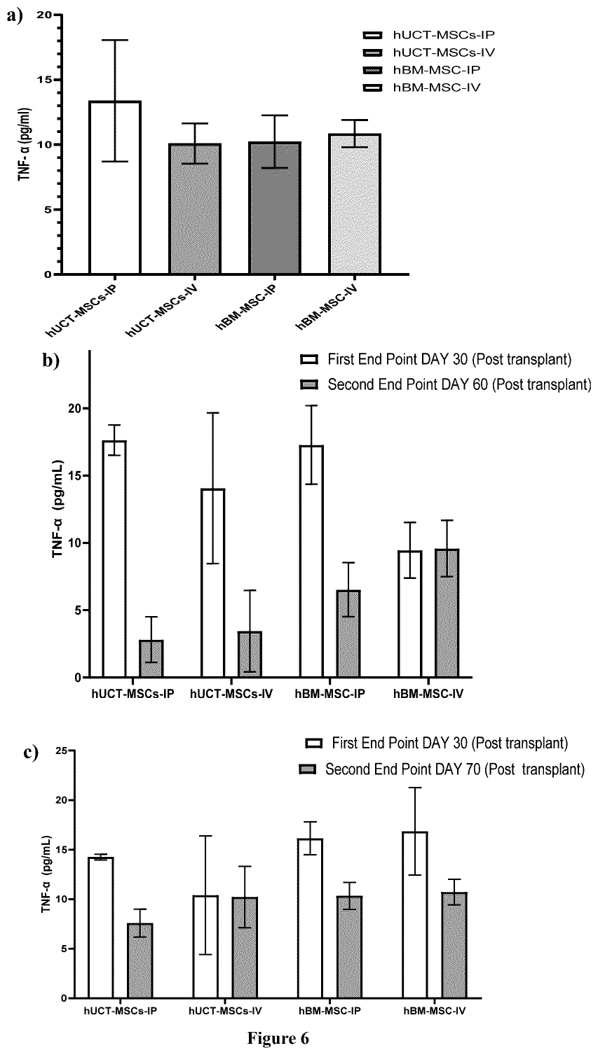

FIG. 6).

In case of moderate group both products 1 and 2 are showing good results in intra plantar mode as well as in intravenous route.

In case of severe group, products 1 and 2 are showing good results in intra venous mode.

TABLE 3a
TNF-α concentration (pg/ml) (Mean ± SE) in the different group of
treatment animals

| | Product → | | | |
|---|---|---|---|---|
| Group | hUCT- | hUCT- | hBM- | hBM- |
| ↓ | MSCs-IP | MSCs-IV | MSC-IP | MSC-IV |
| Mild | 13.38 ± 4.68 | 10.09 ± 1.55 | 10.23 ± 2.03 | 10.85 ± 1.05 |
| (30 Days post- | | | | |
| Transplant) | | | | |
| Moderate | 17.64 ± 1.13 | 14.06 ± 5.6 | 17.28 ± 2.92 | 9.45 ± 2.07 |
| First End Point | | | | |
| (30 Days post- | | | | |
| Transplant) | | | | |
| Moderate | 2.81 ± 1.7 | 3.44 ± 3.03 | 6.52 ± 2.01 | 9.58 ± 2.09 |
| Second End | | | | |
| Point | | | | |
| (60 Days post- | | | | |
| Transplant) | | | | |
| Severe | 14.26 ± 0.28 | 10.40 ± 5.99 | 16.66 ± 1.66 | 16.86 ± 4.41 |
| First End Point | | | | |
| (30 Days post- | | | | |
| Transplant) | | | | |
| Severe Second | 7.59 ± 1.41 | 10.22 ± 3.10 | 10.34 ± 1.36 | 10.72 ± 1.29 |
| End Point | | | | |
| (70 Days post- | | | | |
| Transplant) | | | | |

TABLE 3b
TNF-α quantification in diseased animal and diseased animal treated
with MSCs

| | Disease group | Disease group with | |
|---|---|---|---|
| | with no product | product administered | |
| | administered | (pg/ml) | Improve- |
| | (pg/ml) | diseased animal | ment |
| Disease Severity | diseased animal | treated with MSCs | (%) |
| Mild | 33.67 ± 2.28 | 20.46 ± 4.06 | 39.23% |
| (Day 30 after | | | |
| transplant of MSCs) | | | |
| Moderate | 43.98 ± 0.96 | 18.89 ± 4.15 | 57.04% |
| (Day 30 post- | | | |
| transplant of MSCs) | | | |
| Moderate | 58.63 ± 3.70 | 5.62 ± 3.40 | 90.41% |
| (Day 60 post- | | | |
| transplant of MSCs) | | | |
| Severe | 63.65 ± 2.15 | 20.80 ± 11.90 | 67.32% |
| (Day 30 post- | | | |
| transplant of MSCs) | | | |
| Severe | 162.86 ± 28.08 | 15.18 ± 2.82 | 90.67% |
| (Day 70 post- | | | |
| transplant of MSCs) | | | |

### Detection of IL-10 Level in Blood Serum

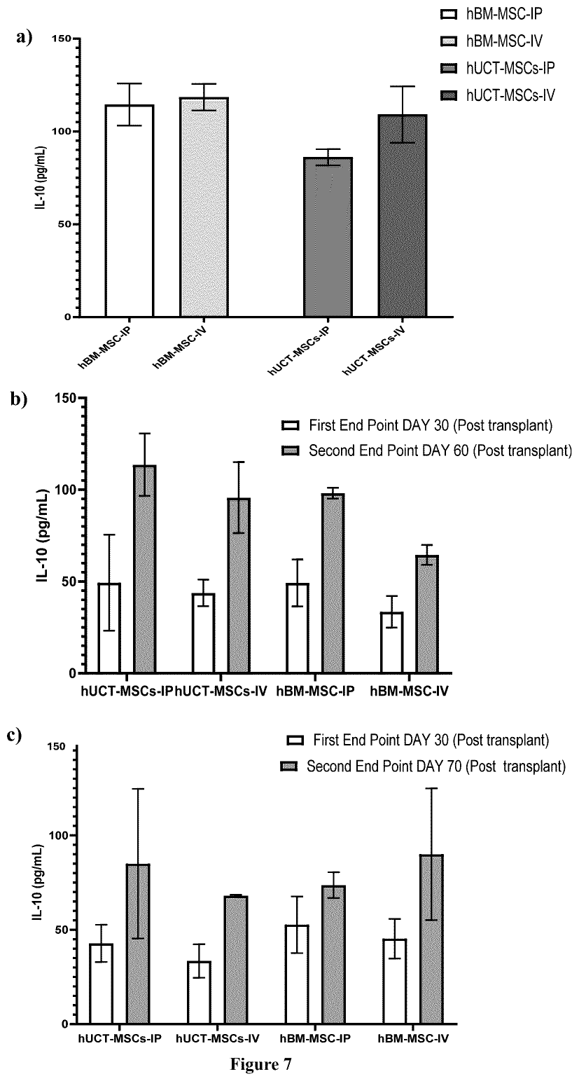

In case of mild group, intravenously injected product 1 and 2 is showing good results by both intravenous as well as by intra plater routes (Table 4a and Table 4b; FIG. 7).

In case of moderate group, product 1 and product 2 is giving good results by intravenous route as well as by intra planter route.

In case of severe group, product 1 is showing good results by both route and Product 2 is giving good results by intravenous route.

TABLE 4a
IL-10 level (pg/ml) (Mean + SE) in the different group of treated animals.

| | Product → | | | |
|---|---|---|---|---|
| ↓ | hUCT- | hUCT- | hBM-MSC- | hBM- |
| Group | MSCs-IP | MSCs-IV | IP | MSC-IV |
| Mild | 86.06 ± 4.37 | 109.09 ± 15.16 | 114.46 ± 11.32 | 118.4 ± 7.2 |
| (30 Days post-Transplant) | | | | |
| Moderate First End Point | 49.40 ± 26.14 | 43.76 ± 7.24 | 49.30 ± 12.79 | 33.50 ± 8.61 |
| (30 Days post-Transplant) | | | | |
| Moderate Second End | 113.62 ± 17.4 | 95.69 ± 19.36 | 98.13 ± 2.96 | 64.52 ± 5.39 |
| Point | | | | |
| (60 Days post-Transplant) | | | | |
| Severe First End Point | 42.83 ± 9.88 | 33.48 ± 8.87 | 52.67 ± 15.03 | 45.29 ± 10.18 |
| (30 Days post-Transplant) | | | | |
| Severe Second End Point | 85.02 ± 39.59 | 68.02 ± 0.52 | 73.67 ± 6.84 | 90.02 ± 34.89 |
| (70 Days post-Transplant)) | | | | |

TABLE 4b
IL-10 quantification in diseased animal amd diseased
animal treated with MSCs

| | Disease group | Disease group with | |
|---|---|---|---|
| | with no product | product administered | |
| | administered | (pg/ml) | Improve- |
| Disease | (pg/ml)diseased | diseased animal | ment |
| Severity | animal | treated with MSCs | (%) |
| Mild | 86.06 ± 4.37 | 118.4 ± 7.2 | 37.57% |
| (Day 30 after | | | |
| transplant of | | | |
| MSCs) | | | |
| Moderate | 33.50 ± 8.61 | 49 30 ± 12.79 | 47.16% |
| (Day 30 post- | | | |
| transplant of | | | |
| MSCs) | | | |
| Moderate | 55.26 ± 1.74 | 113.62 ± 17.4 | 105.60% |
| (Day 60 post- | | | |
| transplant of | | | |
| MSCs) | | | |
| Severe | 33.48 ± 8.87 | 60.89 ± 1.97 | 81.69% |
| (Day 30 post- | | | |
| transplant of | | | |
| MSCs) | | | |
| Severe | 46.60 ± 3.54 | 90.02 ± 34.89 | 93.17% |
| (Day 60 post- | | | |
| transplant of | | | |
| MSCs) | | | |

Overall, serum levels of TNF-α, decreased markedly by both test products compared to G6 (Positive Control) and reference groups. In mild, moderate, and severe group, Levels of IL-10 were seen to increase with both test products.

### 2. Safety:

Mortality and clinical Signs: Test Product 1 and Test product 2 did not cause any mortality and there were no evident clinical signs during the observation period.

Body Weight: Weekly body weights records showed that all the animals gained the body weights normally throughout the experimental period Haematology Analysis: All the haematology parameters including WBC, RBC count HGB and ESR of all the groups were comparable with those of control animals, except there was significant increase in WBC count in reference control group (G7) animals as compared with that of G1 group animals. (Table 5)

TABLE 5
Haematology Parameter Results

| | | | | |
|---|---|---|---|---|
| | WBC | RBC | HGB | ESR |
| Mean/SD/N | (10³/μL) | (10⁶/μL) | (g/dL) | (mm/hr) |

G1

| | | | | |
|---|---|---|---|---|
| Mean | 8.60 | 7.23 | 12.84 | 5.86 |
| SD | 1.76 | 1.10 | 0.10 | 0.69 |
| N | 7 | 7 | 7 | 7 |

G2

| | | | | |
|---|---|---|---|---|
| Mean | 10.32 | 7.82 | 12.66 | 5.17 |
| SD | 2.64 | 1.30 | 2.19 | 1.03 |
| N | 12 | 12 | 12 | 12 |

G3

| | | | | |
|---|---|---|---|---|
| Mean | 10.37 | 7.49 | 12.15 | 5.25 |
| SD | 3.43 | 0.85 | 1.18 | 0.97 |
| N | 12 | 12 | 12 | 12 |

G4

| | | | | |
|---|---|---|---|---|
| Mean | 11.11 | 7.71 | 12.44 | 5.50 |
| SD | 4.96 | 1.13 | 1.88 | 0.80 |
| N | 12 | 12 | 12 | 12 |

G5

| | | | | |
|---|---|---|---|---|
| Mean | 10.38 | 7.36 | 12.28 | 5.58 |
| SD | 4.19 | 0.64 | 0.99 | 0.90 |
| N | 12 | 12 | 12 | 12 |

G6

| | | | | |
|---|---|---|---|---|
| Mean | 10.84 | 7.40 | 13.00 | 5.50 |
| SD | 4.72 | 0.44 | 0.82 | 1.08 |
| N | 10 | 10 | 10 | 10 |

G7

| | | | | |
|---|---|---|---|---|
| Mean | 12.71 | 7.81 | 13.31 | 5.60 |
| SD | 4.56 | 0.43 | 0.70 | 0.97 |
| N | 10 | 10 | 10 | 10 |

Collectively, both the test products proved to be efficacious in CFA induced rheumatoid arthritis treatment. The treatment with test product 1 and test product 2 ameliorated histopathological changes in joints that were caused by treatment of CFA and modulated the expression of cytokines. In consideration of all the efficacy parameters, such as Arthritis score, Haematology, biomarker analysis and histopathological studies confirms that both the test products, viz., hUCT MSC and hBM MSC are found effective in the treatment of CFA induced RA in SD rats. The doses of both the products well tolerated by the animals and product found safe with dose of 1.5×10⁶ and 4×10⁶ cells when injected intra-plantar (IP) and intravenous (IV) route in the animals respectively.

### Advantages of the Present Disclosure

The present disclosure provides a method of treating autoimmune disease using a composition comprising a population of MSCs and a carrier specifically with the following advantages.
1. The disclosed treatment method was effective in treating arthritis.
2. Both intravenous and intra planter routes used for the treatment are giving comparable results in both BM-MSCs and CT-MSCs.
3. Intravenous route of administering the composition is more effective in treating the severe stage of arthritis.
4. Biomarker TNF-α level in treated subject is significantly decreased and at the same time the level of IL-10 is increased, showing good recovery in the arthritis subjects for the disclosed method of treatment.

I/We claim:

1. A method of inducing tissue regeneration, said method comprising administering to said tissue a composition comprising a population of mesenchymal stem cells (MSCs) and a carrier, wherein at least 70% of the MSCs express at least one marker selected from CD73 CD90 or CD105;
and less than 5% of MSCs express CD45 marker and HLA-DR marker (Human Leukocyte Antigen—DR isotype); wherein said population of MSCs is a homogeneous population having a size in the range of 15-30 μm; and wherein said composition comprises secretome of said MSCs having VEGF in an amount in the range of 2050 to 2390 μg per million MSCs, and IL-10 in an amount in the range of 1430 to 1690 μg per million MSCs.

2. The method as claimed in claim 1, wherein said tissue is selected from epithelial tissue; connective tissue like bone tissue, cartilage tissue; muscle tissue, elastic tissue, or nervous tissue.

3. A method of treating an autoimmune disease in a subject in need thereof, the method comprising:
administering a therapeutically effective amount of a composition comprising a population of mesenchymal stem cells (MSCs) and a carrier to the subject by intra-plantar route, intravenous route, intramuscular route, intraarticular route, intraosseous route, or subcutaneous route;
wherein at least 70% of the MSCs express at least one marker selected from CD73 CD90 or CD105; and less than 5% of MSCs express CD45 marker and (Human Leukocyte Antigen-DR isotype) HLA-DR marker;
wherein said population of MSCs is a homogeneous population having a size in the range of 15-30 μm; and wherein said composition comprises secretome of said MSCs having VEGF in an amount in the range of 2050 to 2390 μg per million MSCs, and IL-10 in an amount in the range of 1430 to 1690 μg per million MSCs.

4. The method as claimed in claim 3, wherein the composition is administered in a range of 1 to 5 doses at a time interval of 1 to 2 months; wherein each dose comprises of 0.5 to 10 million cells per kg of the subject's body weight.

5. The method as claimed in claim 3, wherein the method increases the levels of IL-10 in the subject; wherein the increase in levels of IL-10 estimated 30 days post administration of the composition in the subject having mild autoimmune disease is in the range of 30 to 50% as compared to a control;
wherein the increase in levels of IL-10 estimated 30 days post administration of the composition in the subject having moderate autoimmune disease is in the range of 40 to 60% as compared to a control;
and wherein the increase in levels of IL-10 estimated 30 days post administration of the composition in the subject having severe autoimmune disease is in the range of 70 to 90% as compared to a control.

6. The method as claimed in claim 3, wherein the method increases the levels of IL-10 in the subject; wherein the increase in levels of IL-10 estimated 60 days post administration of the composition in the subject having moderate autoimmune disease is in the range of 90 to 120% as compared to a control;
and wherein the increase in levels of IL-10 estimated 60 days post administration of the composition in the subject having severe autoimmune disease is in the range of 80 to 100% as compared to a control.

7. The method as claimed in claim 3, wherein the method decreases the levels of TNF-α in the subject; wherein the decrease in levels of TNF-α estimated in the subject having mild autoimmune disease 30 days post administration of the composition is in the range of 30 to 50% as compared to a control;
wherein the decrease in levels of TNF-α estimated in the subject having moderate autoimmune disease 30 days post administration of the composition is in the range of 50 to 70% as compared to a control; and
wherein the decrease in levels of TNF-α estimated in the subject having severe autoimmune disease 30 days post administration of the composition is in the range of 60 to 80% as compared to a control.

8. The method as claimed in claim 3, wherein the method decreases the levels of TNF-α in the subject; wherein the decrease in levels of TNF-α estimated in the subject having moderate autoimmune disease 70 days post administration of the composition is in the range of 80 to 100% as compared to a control; and wherein the decrease in levels of TNF-α estimated in the subject having severe autoimmune disease 70 days post administration of the composition is in the range of 80 to 100% as compared to a control.

9. The method as claimed in claim 3, wherein said subject having mild autoimmune disease exhibits 95 to 100% recovery of symptoms 30 days post administration of the composition; wherein said subject having moderate autoimmune disease exhibits 55 to 65% recovery of symptoms 60 days post administration of the composition; and wherein said subject having severe autoimmune disease exhibits 35 to 45% recovery of symptoms 60 days post administration of the composition.

10. The method as claimed in claim 3, wherein the mild autoimmune disease is mild arthritis with an arthritis scoring in the range of 1≥ to ≤2, and/or a Disease Activity Score in 28 joints (DAS 28 score) in the range of 2.6≥ to ≤3.1; wherein the moderate autoimmune disease is moderate arthritis with an arthritis scoring in the range of 2≥ to ≤3, and/or a DAS 28 score in the range of 3.1≥ to ≤5.1; and wherein the severe autoimmune disease is severe arthritis with an arthritis scoring in the range of 3≥ to ≤4, and/or a DAS 28 score>5.1.

11. The method as claimed in claim 1, wherein said carrier is selected from serelaxin, Ringer's lactate solution, human serum albumin (HSA), DMEM medium, saline solution, phosphate buffered saine (PBS) buffer, Hank's balanced salt solution (HBSS), human plasma, plasma lysate, human albumin, or mixtures thereof, preferably the carrier comprises of serelaxin, Ringer's lactate solution, human serum albumin (HSA), dextran-40, heparin, hyaluronidase or combinations thereof.

12. The method as claimed in claim 1, wherein the MSCs are derived from umbilical cord tissue, cord blood, adipose tissue, bone marrow, or dental pulp, preferably the MSCs are derived from umbilical cord tissue or bone marrow.

13. The method as claimed in claim 3, wherein the MSCs are autogenic or allogenic to the subject.

14. The method as claimed in claim 3, wherein the auto immune disease is selected from the group of consisting of Rheumatoid Arthritis (RA), Acromegaly, Acquired Aplastic Anemia, Acquired Hemophilia, Agammaglobulinemia, Alopecia Areata, Ankylosing Spondylitis (AS), Anti-NMDA Receptor Encephalitis, Antiphospholipid Syndrome (APS), Arteriosclerosis, Autoimmune Addison's Disease (AAD), Autoimmune Autonomic Ganglionopathy (AAG), Autoimmune Encephalitis (AE)/Acute Disseminated Encephalomyelitis (ADEM), Autoimmune Gastritis, Autoimmune Hemolytic Anemia (AIHA), Autoimmune Hepatitis, Autoimmune Hyperlipidemia, Autoimmune Hypophysitis/Lymphocytic Hypophysitis, Autoimmune Inner Ear Disease (AIED), Autoimmune Lymphoproliferative Syndrome (ALPS), Autoimmune Myelofibrosis (AIMF), Autoimmune Myocarditis, Autoimmune Oophoritis, Autoimmune Pancreatitis (AIP), Autoimmune Polyglandular Syndromes (APS), Autoimmune Progesterone Dermatitis (APD), Autoimmune Retinopathy (AIR), Autoimmune Sudden Sensorineural Hearing Loss, Balo Disease/Concentric Sclerosis, Behçet's Disease, Birdshot Chorioretinopathy/Birdshot Uveitis, Bullous Pemphigoid, Castleman Disease, Celiac Disease, Chagas Disease, Chronic Inflammatory Demyelinating Polyneuropathy (CIDP), Chronic Autoimmune Urticaria, Churg-Strauss Syndrome/Eosinophilic Granulomatosis with Polyangiitis (EGPA), Cogan's Syndrome (CS), Cold Agglutinin Disease (CAD), Crest Syndrome, Crohn's Disease, Stricturing Crohn's Disease, Cronkhite-Canada Syndrome (CCS), Cryptogenic Organizing Pneumonia (COP), Dermatitis Herpetiformis (DH), Dermatomyositis, Diabetes, Type 1 (TID), Discoid Lupus Erythematosus (DLE), Dressler's Syndrome/Post myocardial Infarction/Post pericardiotomy Syndrome, Eczema/Atopic Dermatitis, Eosinophilic Fasciitis, Erythema Nodosum, Essential Mixed Cryoglobulinemia, Evans Syndrome, Fibrosing Alveolitis/Idiopathic Pulmonary Fibrosis (IPF), Giant Cell Arteritis/Temporal Arteritis/Horton's Disease, Giant Cell Myocarditis, Glomerulonephritis (GN), Goodpasture's Syndrome/Anti-Gbm/Anti-Tbm Disease, Granulomatosis With Polyangiitis (GPA)/Wegener's Granulomatosis, Graves' Disease (GD), Guillain-Barrè Syndrome (GBS), Hashimoto's Thyroiditis/Autoimmune Thyroiditis, Henoch-Schölein Purpura (HSP)/Iga Vasculitis, Hidradenitis Suppurativa, Hurst's Disease/Acute Hemorrhagic Leukoencephalitis (AHLE), Hypogammaglobulinemia, Iga Nephropathy/Berger's Disease, Immune-Mediated Necrotizing Myopathy (IMNM), Immune Thrombocytopenia (Itp)/Autoimmune Thrombocytopenia Purpura, Inclusion Body Myositis (IBM), Igg4-Related Sclerosing Disease (ISD), Interstitial Cystitis, Juvenile Idiopathic Arthritis (Jia)/Adult-Onset Still's Disease, Juvenile polymyositis//Juvenile dermatomyositis/juvenile myositis, Kawasaki disease, Lambert-Eaton Myasthenic Syndrome (LEMS), Leukocytoclastic vasculitis, Lichen Planus, Lichen Sclerosus, Ligneous conjunctivitis, Linear Iga Disease (LAD), Lupus Nephritis (LN), Lyme Disease/Chronic Lyme Disease/Post-Treatment Lyme Disease Syndrome (PTLDS), Lymphocytic colitis/microscopic colitis, Lymphocytic hypophystitis/autoimmune hypophystitis, Ménière's Disease, Microscopic Polyangiitis (MPA)/ANCA-Associated Vasculitis, Mixed Connective Tissue Disease (MCTD), Mooren's ulcer, Mucha-Habermann disease, Multifocal motor neuropathy, Multiple Sclerosis (MS), Myalgic Encephalomyelitis/Chronic Fatigue Syndrome (ME/CFS), Myasthenia Gravis (MG), Narcolepsy, Neuromyelitis Optica/Devic's Disease, Ocular Cicatricial Pemphigoid, Opsoclonus-myoclonus syndrome (OMS), Palindromic Rheumatism, Paraneoplastic Cerebellar Degeneration (PCD), Paraneoplastic Pemphigus, Parry-Romberg Syndromeherth (PRS)/Hemifacial Atrophy (HFA)/Progressive Facial Hemiatrophy, Paroxysmal Nocturnal Hemoglobinuria (PNH), Peripheral uveitis/pars planitis, PANS/PANDAS, Parsonage-Turner Syndrome (PTS), Pemphigoid Gestationis (PG), Pemphigus Foliaceus, Pemphigus Vulgaris, Pernicious anemia, POEMS Syndrome, Polyarteritis Nodosa (PAN), Polymyalgia Rheumatica, Polymyositis, Postural Orthostatic Tachycardia Syndrome (Pots), Primary Biliary Cirrhosis (PBC), Primary Sclerosing Cholangitis (PSC), Psoriasis, Palmoplantar Pustulosis (PPP), Psoriatic Arthritis, Pulmonary fibrosis, idiopathic (IPF), Pure Red Cell Aplasia (PRCA), Pyoderma gangrenosum, Rasmussen's encephalitis, Raynaud's Syndrome, Reactive Arthritis, Reflex sympathetic dystrophy syndrome (RSD)/Complex regional pain syndrome (CRPS), Relapsing Polychondritis (RP), Restless leg syndrome (RLS)/Willis-Ekbom disease, Rheumatic Fever, Sarcoidosis, Schmidt Syndrome/Autoimmune Polyendocrine Syndrome Type II, Scleritis, Scleroderma, Sclerosing Mesenteritis/Mesenteric Panniculitis, Serpiginous choroidopathy, Sjögren's Syndrome, Stiff person syndrome (SPS), Small Fiber Sensory Neuropathy (SFSN), Small Fiber Sensory Neuropathy (SFSN), Systemic Lupus Erythematosus (SLE), Subacute bacterial endocarditis (SBE), Subacute cutaneous lupus, Susac's syndrome, Sydenham's Chorea, Sympathetic ophthalmia, Takayasu's arteritis (vasculitis), Testicular Autoimmunity, Tolosa-Hunt syndrome, Transverse myelitis (TM), Tubulointerstitial nephritis uveitis syndrome (TINU), Ulcerative Colitis, Undifferentiated Connective Tissue Disease, Uveitis, Vasculitis, VEXAS Syndrome, Vogt-Koyanagi-Harada syndrome (VKH), Osteoarthritis, AVN, vertebral compression factor, urethral stricture, and ureteric stricture.
